(19) United States
(12) Patent Application Publication
(10) Pub. No.: US 2024/0424042 A1
(43) Pub. Date: Dec. 26, 2024

(54) PHARMACEUTICAL COMPOSITIONS USEFUL FOR THE PREVENTION OR TREATMENT OF VIRAL INFECTIONS

(71) Applicant: BIOCODEX, Gentilly (FR)

(72) Inventors: Marc VERLEYE, Remy (FR); Xavier ROUX, Compiègne (FR); Marie-Emmanuelle LE GUERN, Compiegne (FR)

(21) Appl. No.: 18/696,076

(22) PCT Filed: Sep. 22, 2022
(86) PCT No.: PCT/EP2022/076396

§ 371 Date: Mar. 27, 2024

(30) Foreign Application Priority Data
Sep. 27, 2021 (EP) 21315190.5

(51) Int. Cl. A61K 36/064; A61K 45/06; A61P 31/16

(52) U.S. Cl. CPC A61K 36/064; A61K 45/06; A61P 31/16

(57) ABSTRACT

The present invention relates to a yeast cell-derived product for use for the prevention or treatment of a viral respiratory infection.

### FIELD OF THE INVENTION

The present invention relates to medicaments and pharmaceutical compositions for preventing or treating viral respiratory infections.

### BACKGROUND OF THE INVENTION

Viral respiratory infections are the most frequently occurring diseases in all age groups globally. They are caused by a diverse group of viruses, including coronaviruses, rhinoviruses, respiratory syncytial virus, parainfluenza, and influenza viruses, that target the respiratory tract and result in illnesses ranging from a common cold to severe pneumonia.

Generally, viral respiratory infections result when a virus infects the cells of the respiratory mucosa. This can occur when virus particles are inhaled or directly contact a mucosal surface of the nose or eyes. Infected individuals shed virus into the environment by coughing or sneezing or even during quiet breathing. Virus shed during coughing and sneezing is often present in large droplets that fall out of the air within a short distance. If the virus falls on a surface and survives, it can be transmitted when someone touches the infected surface and then touches their nose, eyes, or mouth. Virus is also spread by the airborne route in the form of small droplet nuclei that can remain suspended for long periods of time and can be inhaled into the lower respiratory tract.

Influenza viruses are spread by contact as well as by airborne transmission, but the mode of transmission of severe acute respiratory syndrome coronavirus 2 (SARS-COV-2) is still being discussed.

When a person is infected with a respiratory virus, there is an incubation period before onset of clinical signs and symptoms. During the incubation period, the virus attaches to and infects cells, replicates its genome, and spreads to infect adjacent cells. The incubation period for influenza is short, typically 1-2 days, whereas for SARS-COV-2, it is 4.5-5.8 days.

Infection control and prevention strategies include hand hygiene, surface decontamination, wearing a mask and respirators. Prevention strategies also include annual vaccination against influenza. However, despite the vaccine, influenza continues to have a major health impact and vaccines for the other respiratory viruses are currently not available.

Current therapies for viral respiratory infections include by way of example oseltamivir (Beigel et al. (2017), Lancet Infect Dis, 17 (12): 1255-1265). However, most of the antiviral therapies cannot be used routinely and often induce side effects.

Accordingly, there is still a need for alternative treatments of viral respiratory infections.

### SUMMARY OF THE INVENTION

The present invention arises from the unexpected finding by the inventors that Saccharomyces boulardii CNCM I-745 supernatant significantly increases cell survival of H1N1 infected cells.

Thus, the present invention relates to a yeast cell-derived product for use for the prevention or treatment of a viral respiratory infection.

In one embodiment, the present invention relates to the yeast cell-derived product for use as defined above for preventing or treating cell death induced by a pulmonary viral infection.

In one embodiment of the present invention, the yeast cell-derived product for use as defined above is in combination with at least one additional compound for the prevention or treatment of a viral respiratory infection.

The present invention also relates to a pharmaceutical composition or medicament comprising a yeast cell-derived product as defined above as active substance, and optionally at least one pharmaceutically acceptable carrier or excipient, for use for preventing or treating a viral respiratory infection.

In an embodiment of the present invention, the above defined pharmaceutical composition or medicament further comprises at least one additional compound useful for the prevention or treatment of a viral respiratory infection.

The present invention also relates to a pharmaceutical composition or medicament comprising:
at least one yeast cell-derived product as defined above as active substance, and
at least one additional compound useful for the prevention or treatment of a viral respiratory infection,
   optionally in association with at least one pharmaceutically acceptable carrier or excipient.

The present invention also relates to a method for preventing or treating a viral respiratory infection in an individual, comprising administering to the individual an effective or prophylactic amount of a yeast cell-derived product as defined above.

In an embodiment of the method as defined above, the yeast cell-derived product is in association with at least one additional compound useful for the prevention or treatment of a viral respiratory infection.

The present invention also relates to the use of a yeast cell-derived product for the preparation of a medicament intended to prevent or treat a viral respiratory infection.

In an embodiment of the use as defined above, the medicament comprises at least one additional compound useful for the prevention or treatment of a viral respiratory infection.

The present invention also relates to products comprising:
a least one yeast cell-derived product as defined above,
at least one additional compound useful for the prevention or treatment of a viral respiratory infection,
   as a combined preparation for simultaneous, separated or sequential use for preventing or treating a viral respiratory infection in an individual.

### DETAILED DESCRIPTION OF THE INVENTION

As intended herein, the word “comprising” is synonymous to “include” or “contain”. When a subject-matter is said to comprise one or several features, it is meant that other features than those mentioned can be comprised in the subject-matter. Conversely, the expression “constituted of” is synonymous to “consisting of”. When a subject-matter is said to consist of one or several features, it is meant that no other features than those mentioned are comprised in the subject-matter.

### Yeast Cell-Derived Product

A “yeast” according to the invention is a fungus, preferably unicellular. The yeast cells according to the invention are preferably of the genus Saccharomyces, more preferably of the species Saccharomyces cerevisiae and even more preferably of the species Saccharomyces cerevisae var boulardii. As intended herein, Saccharomyces cerevisiae var. boulardii is synonymous with Saccharomyces boulardii and is well known to one of skilled in the art and is notably described in Hennequin et al. (2001) J. Clin. Microbiol. 39:551-559.

Particularly preferably, the cells of Saccharomyces boulardii according to the invention are obtained from medicinal products of the brand Ultra-Levure® or from deposits in the American Type Culture Collection (ATCC, USA) under reference 74012 or in the Collection Nationale de Culture et de Microorganismes (CNCM, France) under reference I-745. Preferably also the cells of Saccharomyces boulardii are of Saccharomyces boulardii CNCM 1-745 or Saccharomyces cerevisiae var. boulardii HANSEN CBS 5926.

Preferably, the yeast cells according to the invention are lyophilized, such as Saccharomyces boulardii yeast cells of the brand Ultra-Levure®.

Advantageously, the viability and vitality of yeast cells obtained from lyophilizates are greater than can be obtained with other methods of preservation of yeast cells.

As understood here, “lyophilization” is a method of preservation in which the yeast cells are frozen and are then submitted to sublimation of the frozen water that they contain to give a lyophilizate in the form of dry yeast powder preferably containing less than 2% of water and more preferably less than 1% of water. Preferably, the lyophilized yeast cells are obtained from concentrates of yeast cells. Any type of method of lyophilization of yeast cells known by one of skilled in the art can be used. However, the yeast cells are preferably lyophilized according to the invention by means of the following method of lyophilization:
cultivate the yeast cells in a liquid nutrient medium until the cells reach a stationary phase;
concentrate the cultivated yeast cells and freeze the concentrate;
lyophilize the concentrate.

As intended herein the expression “yeast cell-derived product” relates to any product which can be obtained from yeast cells in themselves or which contains yeast cells secretions. It is preferred that the yeast cell-derived product according to the invention is selected from the group consisting of yeast cells, a yeast cell culture, a yeast cell extract, a yeast cell-conditioned medium and a yeast cell culture supernatant. More preferably, the yeast cell-derived product according to the invention is a yeast cell culture supernatant.

As understood herein, “yeast cells” or “yeast cell culture” include viable or dead yeast cells, intact or in the form of debris. Preferably, at least a proportion of the yeast cells according to the invention or comprised in the yeast cell culture according to the invention are viable, in particular viable and cultivable.

The viability of a yeast cell is defined as the capacity of a yeast cell to multiply. The viability of yeast cells can notably be determined by methylene blue coloration and microscopic observation. The number of viable cells and cultivable cells in a sample can be estimated by determining the number of Colony Forming Units (CFU) contained in the sample.

By way of example, the number of CFU of yeast cells in a liquid sample containing yeasts, such as a yeast cell culture, can be determined by spreading a specified volume of the sample on a solid medium, for example a gel medium, allowing the growth of yeasts, and incubating the solid medium for a period of time, for example 48 h, and at a temperature, for example 30° C., allowing the growth of yeast colonies. The number of colonies relative to the volume spread on the solid medium makes it possible to determine the number of CFU contained in the sample. A detailed protocol for determination of CFU according to the invention is notably described in Toothaker and Elmer (1984) Antimicrobial Agents and Chemotherapy 26:552-556 in the paragraph “Assay for S. boulardii”. Moreover, when the yeast sample is in the form of a solid, for example a lyophilized powder, it is preferred to determine the number of CFU contained in the sample after resuspending a specified mass of the sample in an aqueous solution, notably distilled water or a 0.9% NaCl solution at pH 7.

Preferably, yeast cells or a yeast cell culture according to the invention comprise from 10⁸ UFC/g to 10¹² UFC/g, more preferably, from 2.10⁹ UFC/g to 2.10¹¹ UFC/g.

Numerous methods for preparing yeast cells, a yeast cell culture, a yeast cell extract, a yeast cell-conditioned medium and a yeast cell culture supernatant are well known in the art and are notably described in “Yeast Protocols” (1996) Methods in Cell and Molecular Biology, Ed. Ivor H. Evans, Humana Press.

The yeast cell culture according to the invention can be obtained by any standard method well known to one of skilled in the art for culturing yeast cells and for instance as described in the Example section. By way of example the yeast cell culture according to the invention can be obtained by inoculating a complete liquid culture medium such as yeast extract peptone dextrose (YEPD or YPD) and incubating the medium at 30° C.-37° C. under agitation and aerobic conditions during at least 24 hours, 36 hours, or 48 hours and preferably less than 96 hours.

Yeast cells according to the invention can be obtained from a yeast cell culture medium according to the invention through sedimentation or centrifugation of the yeast cells.

Yeast cell extracts according to the invention can be obtained by any yeast cell fragmentation method known in the art applied to the yeast cell culture or the yeast cells according to the invention, such as autolysis, hydrolysis or autoclaving. In particular, the yeast cells extract according to the invention is selected from the group consisting of a membrane extract, a cytoplasmic extract or a nuclear extract.

Preferably, the yeast cell-conditioned medium according to the invention relates to any medium, such as a liquid cell culture medium, which has been contacted by yeast cells. Preferably, the medium has been contacted by yeast cells for a time sufficient for the yeast cells 20 to have secreted in the medium. By way of example, the medium has been contacted by yeast cells during 1 to 9 days, preferably 1, 2, 3, 4, 5, 6, 7, 8 or 9 days. The conditioned medium according to the invention preferably contains molecules secreted by the yeast cells such as proteins. Preferably, the conditioned medium according to the invention contains no cellular debris.

25 The yeast cell culture supernatant can be obtained by any method well known to one of skilled in the art. The yeast cell culture supernatant according to the invention may notably be obtained by centrifugation of the yeast cell culture according to the invention and taking the supernatant part of the centrifuged culture. The yeast cell culture supernatant according to the invention may also be obtained by filtrating the yeast cell culture according to the invention 30 through a filter retaining yeast cells and recuperating the filtrate.

The yeast cell culture, the yeast cell extract, the yeast cell-conditioned medium and the yeast cell culture supernatant according to the invention may have been subjected to at least one treatment or processing step such as centrifugation, filtration, purification, chromatography, concentration, decantation, heating, pasteurisation, autoclaving, drying, freeze-drying or distillation, in particular filtration, more particularly sterilizing filtration for example with 0.2 μm filters.

In another embodiment, the yeast cell-derived product according to the invention contains living cells that allow a long-lasting action.

In another embodiment, the yeast cell-derived product according to the invention does not contain living cells.

Preferably, the yeast cell-derived product for use according to the invention comprises:
taking yeast cells,
culturing the yeast cells,
obtaining a yeast cell-derived product, and
administering the yeast cell-derived product to an individual.

Preferably, the yeast cell-derived product according to the invention is for an administration or is administered at a dosage of from 0.5×10⁸ to 100×10¹⁰ CFU/kg/d or at a dose of from 0.00125 g/kg/d to 25 g/kg/d.

Preferably, the yeast cell-derived product according to the invention is administered at a dose of from 0.5 g/kg to 10 g/kg, more preferably at a dose of from 1 to 6 g/kg and even more preferably at a dose of about 3 g/kg, in particular once, twice, three times, four times or five times per day.

As one of skill in the art will understand, the quantity of yeast cell-derived product to administer by mass unit (kg) relates to the mass of the individual to whom the yeast cell-derived product is intended to be administered. The quantity of yeast cell-derived product expressed in UFC or in g, refers to the quantity of yeast cells administered where the yeast cell-derived product is yeast cells or a yeast cell culture, or to the quantity of yeast cells from which the yeast cell extract, the yeast cell culture supernatant or the yeast cell conditioned medium is prepared. When the quantity of yeast cell-derived product is expressed in mass unit (g), the yeast cell product is preferably under dried or lyophilized (freeze-dried) form, more preferably under lyophilized form. When the yeast cell-derived product is administered in liquid form, it is preferably administered in a volume of from 1uL to 10 mL, more preferably 100 uL to 2 mL, optionally after prior concentration or dilution.

### Viral Respiratory Infections

The virus that causes the respiratory infection according to the invention can be a RNA or a DNA virus. As intended herein, a RNA virus is a virus which has ribonucleic acid (RNA) as its genetic material. As intended herein, a DNA virus is a virus that has a genome made of deoxyribonucleic acid (DNA) that is replicated by a DNA polymerase.

The virus according to the invention can be a non-enveloped virus or an enveloped virus. As intended herein, an enveloped virus is a virus that has an outer wrapping or envelope. This envelope comes from the infected cell, or host, in a process called “budding off.” During the budding process, newly formed virus particles become enveloped or wrapped in an outer phospholipidic coat that is made from a small piece of the cell's plasma membrane.

Preferably, the virus according to the invention is selected from the group consisting of a virus of the Orthomyxoviridae family, Paramyxoviridae family, Hantaviridae family, Coronaviridae family, Picornaviridae family, Adenoviridae family, Herpesviridae family, Papillomaviridae family, Parvoviridae family.

Preferably, the virus according to the invention is selected from the group consisting of: A negative-sense single-stranded RNA virus:
a Orthomyxoviridae virus
   a Alphainfluenza virus
      an Influenza A virus, in particular, an Influenza A virus subtype selected from the group consisting of H1N1, H5N1, H3N2, H1N2, H6N1, H7N7,H7N3, H9N2, H10N7, and H7N9
   a Betainfluenza virus
      an Influenza B virus
   a Gammainfluenza virus
      an Influenza C virus
a Paramyxoviridae virus
   a Rubulavirus
      a Parainfluenza virus, in particular a Parainfluenza virus 1, a Parainfluenza virus 2, a Parainfluenza virus 3 and a Parainfluenza virus 4
      a Mumps virus
   a Pneumovirus
      a respiratory syncytial virus (RSV)
   a human metapneumovirus (HMTV)
   a Morbillivirus
      a Measles virus
a Hantaviridae virus
   an Orthohantavirus
a Coronaviridae virus
   a Betacoronavirus
   severe acute respiratory syndrome coronavirus 1 (SARS-Cov-1)
      severe acute respiratory syndrome coronavirus 2 (SARS-Cov-2)
      Middle East respiratory syndrome-related coronavirus (MERS-Cov)
A positive-sense single-stranded RNA virus:
   a Picornaviridae
      an Enterovirus
      a Rhinovirus
      a Coxsachievirus
      an Echovirus

A DNA virus:
an Adenoviridae virus
a Herpesviridae virus
   a Herpes simplex virus (HSV)
   a Cytomegalovirus
   Varicella-zoster virus (VZV)
   a Epstein-Barr virus (EBV)
a Papillomaviridae virus
   human Papillomavirus (HPV)
a Parvoviridae
   a bocaparvovirus.

More preferably, the virus according to the invention is selected from the group consisting of Influenza A virus, Influenza B virus, Influenza C virus, Parainfluenza virus, Mumps virus, respiratory syncytial virus, Measles virus, SARS-Cov-1, SARS-Cov-2, MERS-Cov, Rhinovirus, Coxsachievirus, Echovirus and mutants or variants thereof. As intended herein, a “mutant or variant” of a virus as defined above, or of a genomic sequence of a virus as defined above, has a genomic sequence or is a nucleotide sequence which has at least 85%, 90%, 95%, 96% 97%, 98%, 99% or 99,5% identity with the genomic sequence of the virus as defined above.

Preferably also the virus as defined above is a human virus, i.e. a virus which can infect a human.

The viral respiratory infection according to the invention is preferably an infection of the upper respiratory tract, the lower respiratory tract, the nasal mucosa, the nasopharyngeal mucosa, the oropharyngeal mucosa, the bronchi, the alveoli, the lung parenchyma and/or the lung.

As intended herein, preventing or treating a respiratory viral infection in an individual encompasses preventing or treating cell death induced by a viral respiratory infection. As intended herein, preventing or treating cell death encompasses the protection of the cells from the killing by a virus as defined above.

As intended herein, preventing or treating a respiratory viral infection in an individual encompasses preventing or treating the symptoms, disorders, syndromes, conditions or diseases such as a viral pneumonia, in particular a H1N1 pneumonia, a H5N1 pneumonia, a parainfluenza pneumonia (HPIV), a respiratory syncytial virus (RSV) pneumonia, a herpes simplex virus type 1 (HSV1) pneumonia, a cytomegalovirus pneumonia, a varicella pneumonia, an Epstein-Barr virus pneumonia, a human papillomavirus (HPV) pneumonia, a respiratory syncytial virus (RSV) bronchiolitis, a respiratory tract infection from measles, a respiratory tract infection from mumps, a human metapneumovirus (HMTV) pneumonitis, a coxsackievirus respiratory tract infection, an echovirus respiratory tract infection, an enterovirus respiratory tract infection, a rhinovirus infection, a hantavirus pulmonary syndrome (HPS), a severe acute respiratory syndrome in particular a severe acute respiratory syndrome-related coronavirus, more particularly a SARS-Cov-1 infection, a SARS-Cov-2infection, a Middle East respiratory syndrome coronavirus (MERS-COV) infection, a common cold, a pharyngitis, an acute laryngotracheobronchitis (croup), a tracheitis, a tracheobronchitis, and/or a bronchiolitis.

### Individual

Preferably, the individual is a bird, such as a chicken, or a mammal, such as a human, a canine, in particular a dog, a feline, in particular a cat, an equine, a bovine, a porcine, a caprine, such a sheep or a goat, a mustelidae, such as mink, or a camelidae, more preferably the individual is a human.

Preferably, the individual according to the invention is a child being 5 years old or younger, more preferably a child being between 5 years old and 2 years old.

Preferably also, the individual according to the invention is a human adult aged 50 or more, more preferably 60 or more, even more preferably 70 or more and most preferably 80 or more.

Preferably also, the individual as defined above suffers from at least one other disease or condition, in particular selected from immunodeficiency, hypertension, diabetes, in particular diabetes mellitus, more particularly type 2 diabetes, a metabolic syndrome, a cardiovascular disease, in particular ischemic cardiomyopathy, a chronic respiratory disease, a cancer, a cardiopulmonary disease, a renal or hepatic insufficiency or a hemoglobinopathy.

Preferably also, the individual according to the invention is a woman in the 2^{nd} or 3^{rd} trimester of pregnancy.

Preferably also, the individual as defined above is overweight or obese.

According to a usual definition a human individual is considered overweight if its Body Mass Index (BMI, body weight in kg relative to the square of the height in meters) is higher than or equal to 25 kg/m² and less than 30 kg/m² and the individual will be said obese if his BMI is higher than or equal to 30 kg/m². The individual according to the invention may notably present with severe obesity, in particular characterized in human by a BMI higher than or equal to 35 kg/m².

More generally, it is preferred that the individual as defined above is a human and has a BMI higher than or equal to 25 kg/m², 26 kg/m², 27 kg/m², 28 kg/m², 29 kg/m², 30 kg/m², 31 kg/m², 32 kg/m², 33 kg/m², 34 kg/m², 35 kg/m² or 40 kg/m².

Besides, the individual as defined above may also have an abdominal obesity, corresponding in particular to a visceral adipose tissue excess. According to a usual definition a male human individual has an abdominal obesity if the abdominal perimeter is higher than or equal to 94 cm, in particular higher than 102 cm and a female human individual has an abdominal obesity if the abdominal perimeter is higher than or equal to 80 cm, in particular higher than 88 cm. The abdominal perimeter measure is well known to one of skilled in the art: abdomen circumference is thus preferably measured midway between the last floating rib and the top of the iliac crest in a standing individual in gentle expiration.

It is particularly preferred that the individual as defined above is a man and presents with an abdominal perimeter higher than or equal to 90 cm, 91 cm, 92 cm, 93 cm, 94 cm, 95cm, 96 cm, 97 cm, 98 cm, 99 cm, 100 cm, 101 cm or 102 cm. It is also preferred that the individual according to the invention is a woman and presents with an abdominal perimeter higher than or equal to 75 cm, 76 cm, 77 cm, 78 cm, 79 cm, 80 cm, 81 cm, 82 cm, 83 cm, 84cm, 85 cm, 86 cm, 87 cm, or 88 cm.

### Additional Compound

Preferably, the other compound suitable for the prevention or treatment of a viral respiratory infection can be selected from any antiviral compound known to the person skilled in the art.

Preferably, the other compound suitable for the prevention or treatment of a viral respiratory infection is selected from the group consisting of oseltamivir, zanamivir, peramivir, baloxavir, ribavirin, a guanosine analog, palivizumab, atorvastatin, cerivastatin, fluvastatin, lovastatin, mevastatin, pitavastatin, pravastatin, rosuvastatin, simvastatin, chloroquine, hydroxychloroquine, azithromycin, remdesivir, penciclovir, favipravir, a cysteine protease inhibitor, in particular a cathepsin L inhibitor, such as camostat and nafamostat, nitazoxanide, thalidomide, fingolimod, carrimycin, lopinavir/ritonavir, methylprednisolone, dexamethasone, bevacizumab, tocilizumab, sarilumab, N-acetylcysteine, recombinant human interferon α1β, arbidol, eculizumab, darunavir, cobicistat, meplazumab, danoprevir, peginterferon alfa-2a, oseltamivir, nicotine, chlorpromazine, intravenous immunoglobulins, a statin, an angiotensin-converting enzyme inhibitor (ACEI)/angiotensin II receptor blocker (ARB), such as losartan, a calcium channel blocker (CCB), such as amlodipine besylate, an amino-bisphosphonate, such as zoledronic acid, and pharmaceutically acceptable salts, esters, hydrates, derivatives, prodrugs or metabolites thereof and/or combinations thereof.

Preferably, the yeast cell-derived product according to the invention, in particular the Saccharomyces boulardii yeast cell-derived product, is not administered with a glucan oligomer, in particular a glucan oligomer from Saccharomyces cerevisiae. More preferably, the yeast cell-derived product according to the invention, in particular the Saccharomyces boulardii yeast cell-derived product, is not administered with a β-glucan, in particular a β-glucan from Saccharomyces cerevisiae.

In an embodiment, the yeast cell-derived product according to the invention, in particular the Saccharomyces boulardii yeast cell-derived product, is not administered with another probiotic. More preferably, the yeast cell-derived product according to the invention, in particular the Saccharomyces boulardii yeast cell-derived product, is not administered with a probiotic selected from the group consisting of Lactobacillus acidophilus, Lactobacillus bulgaricus, Bifidobacterium lactis, Bifidobacterium animalis, Lactobacillus casei, and Lactobacillus reuteri.

### Pharmaceutical Composition

The yeast cell-derived product according to the invention, optionally combined with at least one additional compound useful for the prevention or treatment of a viral respiratory infection, can be comprised in a pharmaceutical composition which can comprise at least one pharmaceutically acceptable vehicle or excipient.

As intended herein “pharmaceutically acceptable vehicle or excipient” refers to any material suitable with a pharmaceutical composition. Preferably, the pharmaceutically acceptable carrier or excipient according to the invention is suitable for a yeast cell-derived product according to the invention in the form of a solid, a liquid or an aerosol.

Preferably, the pharmaceutically acceptable carrier or excipient according to the invention is suitable for an administration by the oral route, the parenteral route, the intradermal route, the intravenous route, the arterial route, the intramuscular route, the nasal route, the rectal or the subcutaneous route.

The pharmaceutically acceptable vehicle or excipient can be selected from dispersants, solubilizers, stabilizers, preservatives, wetting agents etc.

Preferably, the pharmaceutically acceptable carrier or excipient according to the invention, includes but is not limited to any of the standard carrier or excipient known to one of skilled in the art such as water, glycerin, alcohol, oil emulsion, water emulsion, buffered saline solution, sucrose, lactose, starch, methylcellulose, hydroxymethylcellulose, carboxymethylcellulose, croscarmellose sodium, lactose monohydrate, magnesium stearate, microcrystalline cellulose, povidone, sodium lauryl sulfate, mannitol, gelatin, lactose, vegetable oils, acacia gum, liposomes, etc.

### Administration

As intended herein, “combined” or “in combination” means that the yeast cell-derived product, as defined above, is administered at the same time than another compound or product, either together, i.e. at the same administration site, or separately, or at different times, provided that the time period during which the yeast cell-derived product, as defined above, exerts its effects on the individual and the time period during which the additional agent or product exerts its pharmacological effects on the individual, at least partially intersect.

Preferably, the yeast cell-derived product according to the invention is administered in a prophylactically or therapeutically effective amount for preventing or treating a viral respiratory infection.

Preferably, also the yeast cell-derived product according to the invention is administered in a prophylactically or therapeutically effective amount for preventing or treating cell death induced by a viral respiratory infection.

The administration of the yeast cell-derived product or the pharmaceutical composition or medicament comprising the yeast cell-derived product according to the invention can proceed by any method known in the art.

Preferably, the yeast cell-derived product or the pharmaceutical composition or the medicament comprising the yeast cell-derived product according to the invention is in a form suitable for being administered or is administered subcutaneously, intravenously, intramuscularly, intra-dermally, topically or by the oral route, the parenteral route, the intradermal route, the intravenous route, the arterial route, the intramuscular route, the nasal route, the rectal or the subcutaneous route.

Preferably, the yeast cell-derived product or the pharmaceutical composition or medicament comprising the yeast cell-derived product according to the invention is in the form of a powder, a sachet, a tablet, a capsule, a patch, or a liquid or gel solution or an aerosol.

The invention will be further explained by the following Figures and non-limiting Examples.

### DESCRIPTION OF THE FIGURES

FIG. 1

FIG. 1 represents the percentage of survival of cells after H1N1 PR8 infection (black column), of uninfected control cells (column with vertical bars), of H1N1 PR8 infected cells post-treated with 250 μg/ml of S. boulardii CNCM I-745 (column with horizontal bars) and of infected cell post-treated with 500 μg/ml of S. boulardii CNCM I-745 (column with dots). One-way ANOVA+Dunnett's test **p<0.01, ***p<0.001 vs H1N1, n=4.

FIG. 2

FIG. 2 represents the observation of the cytopathic effect of non-infected cell (A), H1N1 PR8 infected cells (B) and H1N1 PR8 infected cells treated with S. Boulardii CNCM I-745 supernant at 125 μg/mL (C), 250 μg/mL (D) and 500 ug/mL (E) using phase contrast microscopy 10X.

Example 1

The inventors have assessed the cell viability effect of S. boulardii CNCM I-745 in a H1N1 PR8Influenza in vitro infection model.

### 1. Material and Method

1.1. Compounds
Test compound: S. boulardii CNCM I-745 supernatant prepared in non-supplemented MEM.
Vehicle: MEM

1.2. Cells Preparation

A549 cells were cultured in minimal essential medium (MEM) supplemented with 10% FBS, 1% pen/strep, 25 mM 4-(2-hydroxyethyl)-1-piperazineethanesulfonic acid (HEPES) buffer and 0.2% bovine serum albumin faction V (BSA). Two distinct cultures were prepared in flasks and incubated at 37°° C., 5% CO2.

1.3. Cell Infection

A total of 10000 A549 cells in 100 uL were seeded per well (in duplicate from each culture) of three 96-well flat-bottom plate. The cells were incubated at 37° C., 5% CO2 untill 90% confluency. Culture medium was removed and replaced by 50 μl of fresh culture medium containing 20% FBS, 2% pen/strep, 50 mM HEPES and 0,4% BSA

Three sample dilutions (1000 μg/mL and 500 μg/mL and 250 μg/mL) were prepared by diluting the test sample in non-supplemented MEM.

Cells were infected with H1N1 PR8 virus (except for uninfected control cells) at MOI of 0.01diluted in serum-free MEM. After 1h absoprtion period, the virus-containing medium was removed and the wells were washed with 50 ul of PBS. Then 50 ul of fresh culture medium containing 20% FBS, 2% pen/strep, 50 mM HEPES and 0.4% BSA wer added to the wells. 50uL of test compound were added for post treated cells.

The plates were then incubated for 72 additional hours at 37°° C., 5% CO2. Culture medium contained 100 ul MEM supplemented with 10% FBS, 1% pen/strep, 25 mM HEPES and 0.2%

BSA. In parallel, 2 types of controls were performed:
1. Uninfected controls
2. Non treated infected controls

A Celltiter-glo assay was performed to characterize viability of infected cells of the second plate.

### 2. Results

2.1. S. Boulardii CNCM I-745 supernatant activity results

2.1.1. Cell Viability

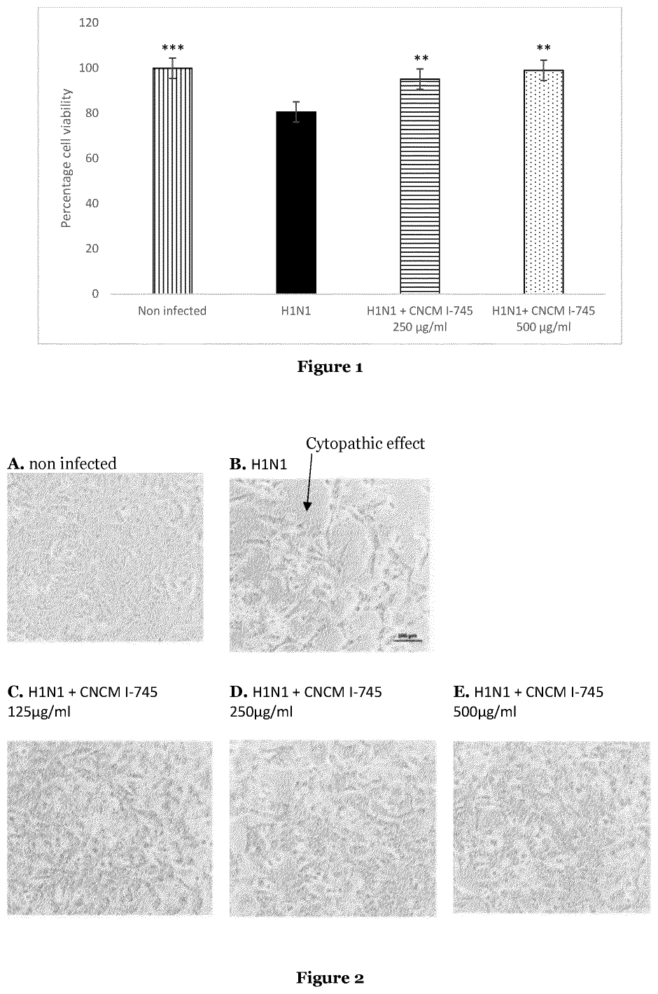

The results are shown in FIG. 1. Cell viability was significantly decreased in H1N1 infected control cells compared to non-infected cells (−19.3%; p<0.001). Moreover, S.boulardii CNCM I-745 supernatant post treatments at 250 μg/mL and 500ug/ml significantly reduced the effects of H1N1 infection by increasing cell viability of +18% and +23% (p<0.01 and p<0.001) respectively compared to H1N1 infected control cells.

2.1.2. Cytopathic Effect

In FIG. 2, it can be observed that in H1N1 PR8 infected cells, a cytopatic effect leading to structural changes can be observed. It has been observed that there is no cytopathic effect in H1N1 PR8 infected cells post-treated with S. boulardii CNCM I-745 supernant at 125 ug/mL, 250 ug/mL and 500 ug/mL.

2.2. Conclusion Post-treatment with S. Boulardii CNCM I-745 supernatant at 250 μg/mL and 500 μg/mL significantly increases H1N1-infected cell survival in comparison to HIN1 infected cells. This effect is correlated with a diminution of cytopathic effect.

1. A method for preventing or treating a viral respiratory infection in an individual, comprising administering a prophylactically or therapeutically effective amount of a yeast cell-derived product to the individual.

2. The method according to claim 1, for preventing or treating cell death induced by a viral respiratory infection.

3. The method according to claim 1, wherein the yeast cell-derived product is selected from the group consisting of yeast cells, a yeast cell culture, a yeast cell extract, a yeast cell-conditioned medium and or a yeast cell culture supernatant.

4. The method according to claim 1, wherein the yeast cell-derived product is derived from yeast cells of the genus Saccharomyces.

5. The method according to claim 1, wherein the yeast cell-derived product is derived from yeast cells of the species Saccharomyces boulardii.

6. The method according to claim 1, wherein the yeast cell-derived product is prepared from lyophilized yeast cells.

7. The method according to claim 1, wherein the yeast cell-derived product is administered orally, rectally, topically, nasally, parentally, intradermally, intravenously, arterially, intramuscularly, or subcutaneously.

8. The method according to claim 1, wherein the yeast cell-derived product is administered at a dosage of from 0.00125 g/kg/d to 25 g/kg/d.

9. The method according to claim 1, wherein the yeast cell-derived product is administered in combination with at least one additional compound useful for the prevention or treatment of a viral respiratory infection.

10. A method for prevention or treatment of a viral respiratory infection in an individual comprising administering a pharmaceutical composition or drug comprising a yeast cell-derived product according to claim 1 as an active substance, and optionally at least one pharmaceutically acceptable carrier or excipient, to the individual.

11. The method according to claim 10, for preventing or treating cell death induced by a viral respiratory infection.

12. The method according to claim 10, wherein the yeast cell-derived product in a dosage of from 50 to 250 mg.

13. The method according to claim 10further comprising administering at least one additional compound useful for the prevention or treatment of a viral respiratory infection, to the individual.

14. The method according to claim 1, wherein the virus is selected from the group consisting of a virus of the Orthomyxoviridae family, Paramyxoviridae family, Hantaviridae family, Coronaviridae family, Picornaviridae family, Adenoviridae family, Herpesviridae family, Papillomaviridae family, and Parvoviridae family.

15. The method according to claim 1, wherein the virus is selected from the group consisting of Influenza A virus, Influenza B virus, Influenza C virus, Parainfluenza virus, Mumps virus, Respiratory syncytial virus, Measles virus, SARS-Cov-1,SARS-Cov-2, MERS-Cov, Rhinovirus, Coxsachievirus, Echovirus and mutants or variants thereof.

16. A pharmaceutical composition comprising as an active substance:
at least one yeast cell-derived product, and
at least one additional compound useful for the prevention or treatment of a viral respiratory infection,
optionally in association with one pharmaceutically acceptable carrier or excipient.

17. A product comprising:
at least one yeast cell-derived product, and
at least one additional compound useful for the prevention or treatment of a viral respiratory infection,
as a combined preparation for simultaneous, separate or sequential use.
